(19) United States
(12) Patent Application Publication
(10) Pub. No.: US 2006/0276806 A1
(43) Pub. Date: Dec. 7, 2006

(54) Instrument for cleaning and rejuvenating the skin by means of non-traumatic abrasion in a controlled and adjustable vacuum

(76) Inventor: Edgardo Luis Martinez Zunino, Guadalajara (ES)

Correspondence Address: BLANK ROME LLP, 600 NEW HAMPSHIRE AVENUE, N.W., WASHINGTON, DC, 20037, US

(21) Appl. No.: 10/556,529

(22) PCT Filed: Apr. 15, 2004
(86) PCT No.: PCT/ES04/00163

§ 371 Date: Nov. 14, 2005

(30) Foreign Application Priority Data
May 20, 2003 (AR) P030101752

(51) Int. Cl. A61B 17/50

(52) U.S. Cl. 606/131

(57) ABSTRACT

This tool includes a headstock having a first body (5) with a passage or channel (7) communicating with a vacuum source. This passage (7) within the first body (5) is branched into a plurality of channels (8) placed downstream in a circular disposition crown-wise around an axis at the lower end (9) of the first body. Around this first body is placed a second body shaped as an annular sleeve-like body (10), being the relative variable height of the second body (10) adjustable in relation to the lower end of the first body. Between the inner surface of this sleeve-like second body (10), the lower end part of the first body (15) and the portion of skin (20) against which this tool is applied is defined an annular air-tight chamber with a variable volume and variable absorption capacity. The lower annular rim (14) of the second body (10) and the lower surface (15) of (9) is pressed against the skin (20) of the user, creating an abrasive action and a detritus removal by aspiration, providing the annular edge (14) rounded edges capable to smooth the skin, while the inner abrasive surface provides an abrasive action over the patch of smoothed skin, advancing the tool over the skin in a continues motion without jumps providing a less irritating abrasive action.

### FIELD OF APPLICABILITY OF THIS INVENTION

This instant invention provides a tool to perform a controlled superficial cleansing of the skin and defects removal thereof, rejuvenating areas of the epidermis of the user undergoing this skin treatment, providing to remove thin outer layers of aged or blemished skin, in order to correct defects, scars, or simply to remove aged surface layers of said skin.

### KNOWN PRIOR ART

The expedient to rub an abrasive material over the surface of the skin is a widely used means in order to cleanse and even correct skin imperfections, being this performance well known through the ages.

Recently U.S. Pat. No. 6,241,739, issued to Waldron, shows a hollow stem connected at one end thereof to a source of vacuum, while its opposite end is connected to a headstock coated with an abrasive material, having one or more central passages. When this abrasive headstock is placed over the skin, this or these inner passages creates a central suction zone which collects the detritus and skin cells removed from the skin by said abrasive headstock, being said removed cells absorbed and further conveyed through said hollow stem as a result of the applied vacuum.

In order to avoid unnecessary repetitions regarding this prior art, U.S. Pat. No. 6,241,739 is hereby introduced in full into this instant specification, wherein it is made mention of additional prior art to said US Patent and the problems to be found in the abrasive skin cleansing process.

The abrasive tool or device shown in this U.S. Pat. No. 6,241,739 consists in a body having a contact surface with the skin coated with an abrasive material which has a plurality of micro-cutting edges, such as a diamond powder, or crystals of an adequate hardness.

This same prior art makes allowance for a contact surface milled with a plurality of hardened micro-grooves offering a plurality of cutting edges.

Even though the above prior art tool performs satisfactorily, at the same time some inconveniences have been found affecting its overall performance, as per the following brief detail:
a) The prior art tool has a central bore, surrounded by abrasive material, and through this central bore it is applied the vacuum for the detritus removal. This implies that the skin of the patient instead of providing a plane and stretched surface, it is pulled and wrinkled against said abrasive material when this tool travels over the skin surface, hence the cutting edges provided by this tool tends to become aggressive, which may lead to a cutaneous irritation in case of sensitive skins, or in skin sensitive zones, or both, with disagreeable consequences.
b) This abrasive coated tool has no way to regulate the height of said abrasive material in relation to the surface of the skin to which it is being applied, while the degree of vacuum thus applied over the surface of said skin may be only regulated through valves or checkpoints operating on its vacuum circuit, or directly modifying the variable working conditions of the vacuum pump or its driving motor, being all the above devices an unwelcome complication which adds to the overall cost of the device.
c) In this prior art abrasive tools, the progress of the abrasive headstock over the zone of the skin under treatment is being performed sliding the abrasive surface of said tool over the skin, which tends to increase its irritation, since said progress is always performed by directly pressing and sliding micro-cutting edges over the epidermis.

### OBJECTS OF THIS INSTANT INVENTION

It is a main object of this instant invention to provide a tool or headstock suitable for performing skin cleansing and skin defects correction by means of abrasion, defining a smooth back thereof which has no abrasive material, providing a first contact of said tool for its advancement against said skin, capable of stretching a smooth area of skin under treatment with said tool, offering to the cutting edges of the abrasive material a portion of skin tangent to said cutting edges, providing a non irritating contact between said tool and the skin.

It is also an object of this invention to have this abrasive material placed over a convex surface, central to said smooth back of the tool, determining said smooth back the first contact between said tool and said skin allowing a non traumatic stretching of this small portion of skin and a smooth progress of this tool over said skin.

Another of this invention is to enable this tool to perform an annular vacuum against its smooth back and the portion of skin against which it is applied, being this contact free of any abrasive material.

It is a further object of the invention a tool capable of stretching and rendering smooth the zone of skin against which it is applied with a portion of said tool having a friction coefficient smaller than the friction coefficient belonging to the part of said tool performing said abrasion and micro-cutting process.

Last, it is also an object of this invention that its part thereof sporting said abrasive surface has a variable height regarding the surface zone of skin against which it is being applied, thus regulating the amount of vacuum applied against said skin by said tool, avoiding unnecessary and costly vacuum regulation means acting on the vacuum circuit.

### BRIEF SUMMARY OF THE INVENTION

TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS.

This tool includes a stem linked at its lower end to a headstock which has an abrasive surface on at least part of its lower end designed to be applied over a surface or zone of skin under abrasion treatment, and a suction zone in said headstock in communication with a source of vacuum, characterised in that said headstock has a first body whose upper end is united to said stem, having this upper end of said first body a passage or channel communicating with a vacuum source; this main passage derives into a plurality of channels in a circular disposition in a lower part of said first body and within said headstock; upstream of the lower outlet of these channels this first body is linked to a second body which defines an annular sleeve-like body having a coaxial axis with the lower end part of said first body; the lower annular rim of said second body has rounded smooth edges, while said lower end of the first body is placed within said annular rim and it has a convex abrasive lower surface; this lower annular rim of said second body has a variable height adjustable in relation to the tangent of said convex surface of said lower end of the first body; an annular air-tight chamber in communication with said passage and said vacuum source is defined between the inner surface of this sleeve-like second body, said lower end part of the first body and the portion of skin against which this tool is applied, being the volume of this chamber and the degree of vacuum applied over said skin variable according to the height adjustment of said second body in relation to said tangent to the lower end of this first body, having this second body means capable to stop and maintain its axial travel in regards to said first body.

### DESCRIPTION OF THE PREFERRED EMBODIMENTS OF THIS INSTANT INVENTION

In order to exemplify the preferred constructions for this invention, it is enclosed the following drawings depicting said embodiments, in accordance to the following description thereof, being said drawings and description given as some of the several possible embodiments for this same invention, hence it must not be assigned to said drawings and description thereof any restrictive purpose, being included within this instant invention all the equivalent means having same function, while the actual scope of this invention is given by the first claim in the following claims chapter.

Also, in all the following drawings, the same reference applies to the same means or means equivalent to the ones depicted.

FIG. 1 depicts a longitudinal cross-section of an embodiment for said headstock, applied to a hollow stem through which the vacuum is applied;

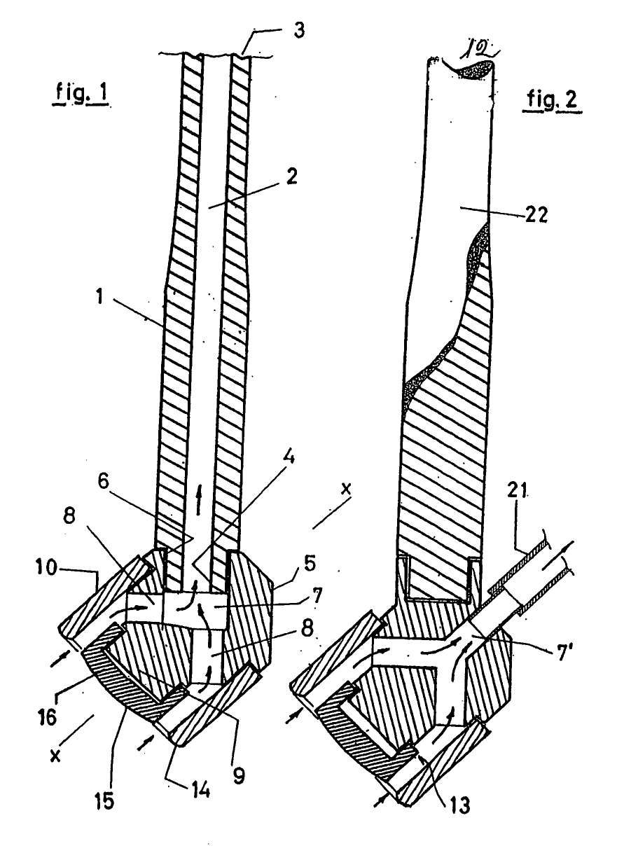

FIG. 2 shows the same cross-section for the headstock connected to an aspiration conduit placed outside of said stem;

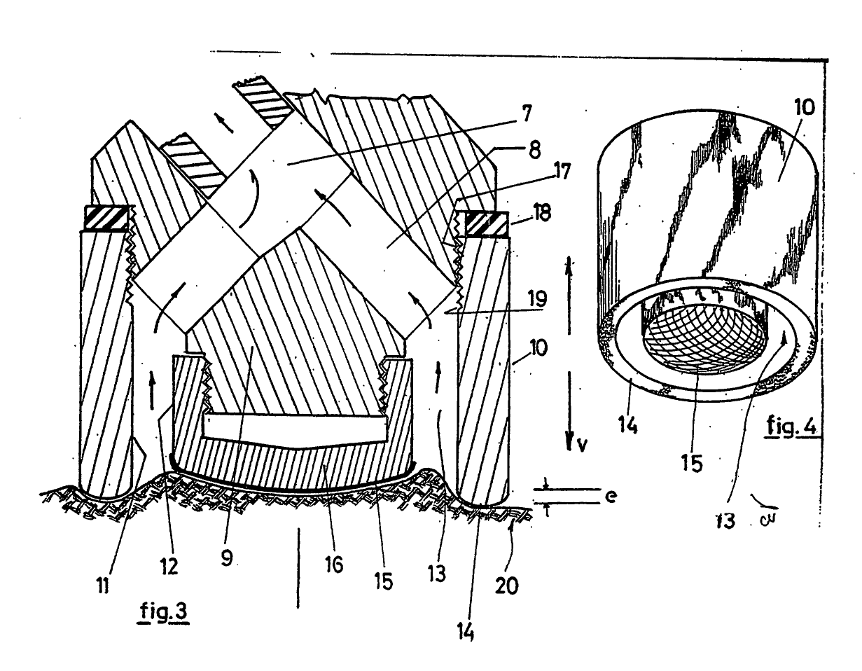

FIG. 3 illustrates a detail of the vertical cross-section of this headstock, as applied over a portion of the skin of the user; and

FIG. 4 shows the lower end portion of said headstock, in a lateral lower perspective and isolated from all the other acting components.

In FIG. 1, reference (1) is a hollow stem, defining an inner conduit (2). The upper end (3) of this conduit (2) communicates with a vacuum pump (not shown). The lower end (4) of said stem (1) is linked to a first body (5) through known and common knowledge means, such as a helical thread or a snap coupling (6). This first body (5) has an inner passage or channel (7) which connects with conduit (2) within said stem. This channel (7) derives into more than two lower passages (8) placed within said first body (5) and opening in a circular or crown disposition around a lower end portion (9) of (5), around its longitudinal axis XX.

Coaxial to said axis XX of this lower end portion (9) and connected thereto it is placed a second body constituted by a sleeve (10), determining between its inner surface (11) and the lateral surface (12) of said lower part (9) of the first body (5) an annular space (13) in communication with the outlet of said branched passages or channels (8) placed crown-wise at the beginning of said lower part (9). This annular chamber (13) communicates through (7) with the conduit in the tube or stem (2).

The lower edge (14) of the second body or sleeve (10) is preferably smooth and rounded, while said lower body (9) is placed coaxial and inner to said smooth rim and has at its lowermost end an abrasive surface (15). This abrasive surface may be obtained by coating or depositing over said lower surface (15) a layer of an abrasive powder, made of crystals capable of providing a plurality of micro-cutting edges, such as a diamond powder according to the teaching in said U.S. Pat. No. 6,241,739 patent, or also, as shown in the Figures of this instant invention, said surface (15) is secured over an additional body (16) or insert which is then affixed on said lower end of the lower body (9), being this insert selectively removable. This surface (15) is convex, and it abrasive condition can be also achieved machining over its convex surface a plurality or grooves and then cementing this part.

One of the main characteristics of this invention is given by the fact that this second body (10) is axially movable over its seat (17) (see FIG. 3) with respect to the first body (5) and it is selectively fixed in a determined position in which its lower edge (14) may be placed tangent to the convex surface of (15) with a difference in height (e) in respect of said surface (15) selected according to the skin sensibility of the user, of the magnitude of the vacuum in said chamber (13) to be attained over said portion of skin and to the depth of the abrasion necessary according to the desired skin cleansing and the zone of the skin being worked upon. The means to fix the relative height (e) of the second body (10) can be of a varied nature, according to the teaching in the art. For instance, the upper end of (10) can be divided in a plurality of parallel prongs and a co-operating lock-nut (not illustrated) pressing the upper end of said prongs of (10) against the surface (17), or they can be made of a ring (18) placed as a stop shoulder limiting the axial displacement of (10), or they can consist in very tight-fitting co-operating screw threads (17-19) which impedes the displacement of (10) without the benefit of a special or adapted tool capable of rotating and thus relatively axially displace said bodies. This axial displacement is indicated by the arrow “v” in FIGS. 3 and 4.

Another characteristic of this instant invention is depicted in FIG. 3, and its is given by the performance of the adjustable height end edge or rounded end shoulder (14). This rounded end shoulder (14) smoothes the zone of skin (20) of the user over which this device is applied, and allows said area of skin facing the abrasive area to be tangentially stretched in regards to said convex surface (15).

This skin cleansing tool, or parts thereof, can be manufactured in a variety of materials, such as chrome steel, plastic material, aluminium, etc., or their combinations. For instance, the second body (10) may be made in a nylon polymer, or Teflon, providing a differential friction coefficient over the skin between said second body and said abrasive area.

In FIG. 2 is observed another construction of this instant invention, in which the collecting channel (7′) acting as a manifold for the branched channels (8) is left open over an upper end of said first body (5), wherein an outer flexible conduit (21) communicates the stem (22) to the vacuum pump (not shown), therefore in place of the stem (1) with its inner channel (2), the first body of this device is connected to this solid rod stem.

In FIGS. 1 and 2, it is indicated by means of arrows the vacuum trajectory from said chamber (13) up to its engagement to the vacuum pump.

1. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, including this tool a stem linked at its lower end to a headstock, having this headstock an abrasive surface on at least part of its free end to be applied over the surface of the zone of skin under abrasion treatment, and a suction zone in said headstock in communication with a source of vacuum, characterised in that said headstock has a first body whose upper end is united to said stem, having this upper end of said first body a passage or channel communicating with a vacuum source; downstream from said passage this main passage derives into a plurality of channels in a circular disposition in a lower part of said first body and within said headstock; upstream of the lower outlet of these channels this first body is linked to a second body which defines an annular sleeve-like body having an axis coaxial with the lower end part of said first body; the lower annular rim of said second body has rounded smooth edges, while said lower end of the first body is placed coaxial within said annular rim and it has a convex abrasive lower surface; this lower annular rim of said second body has a variable height adjustable in relation to the tangent of said convex surface of said lower end of the first body; between the inner surface of this sleeve-like second body, said lower end part of the first body and the portion of skin against which this tool is applied defines an annular air-tight chamber in communication with said passage and said vacuum source, being variable the volume of this chamber and the degree of vacuum applied over said skin according to the height adjustment of said second body in relation to said tangent to the lower end of this first body, having this second body means capable to stop and maintain its axial travel in regards to said first body.

2. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, according to claim 1, characterised for being the passage or channel in said first body in communication with a conduit placed within a hollow stem, in communication with a vacuum pump.

3. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, according to claim 1, characterised by a passage obtained within said hollow stem in communication with a conduit placed outside said stem, being said first body attached to said stem and in communication with a vacuum pump.

4. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, according to claim 1, characterised by an abrasive surface obtain in an insert attached to the lower end of the lower portion of said first body, being this insert selectively removable, while this abrasive surface is obtain by depositing micro-powdered diamond chips coated over the lower outer surface of said insert.

5. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, according to claim 1, characterised by having fixing means fixing the relative position of the axial displacement of said second body and said first body constituted by an annular supplementary ring placed as a stop shoulder against which said second body adjusts pressing against said first body.

6. TOOL FOR CLEANSING AND REJUVENATE THE EPIDERMIS THROUGH NON TRAUMATIC ABRASION PERFORMED UNDER ADJUSTABLE CONTROLLED VACUUM CONDITIONS, according to claim 1, characterised by a smooth lower edge in said second body having better sliding properties than said inner abrasive surface, being this second body manufactured in a low friction polymer material.
